(19) United States
(12) Patent Application Publication
(10) Pub. No.: US 2002/0026087 A1
(43) Pub. Date: Feb. 28, 2002

(54) Process for preparing di-iso-butanes, di-iso-butenes and di-n-butenes from field butanes

(75) Inventors: Franz Nierlich, Marl (DE); Paul Olbrich, Haltern (DE); Wilhelm Droste, Marl (DE); Richard Mueller, Marl (DE); Walter Toetsch, Marl (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: OXENO Olefinchemie GmbH, Marl (DE)

(21) Appl. No.: 09/917,885

(22) Filed: Jul. 31, 2001

(30) Foreign Application Priority Data
Jul. 31, 2000 (EP) 00116504.2

(51) Int. Cl. C07C 6/00; C07C 2/06

(52) U.S. Cl. 585/329; 585/330

(57) ABSTRACT

Butene oligomers and di-iso-butane are prepared from field butanes by a process, which comprises:

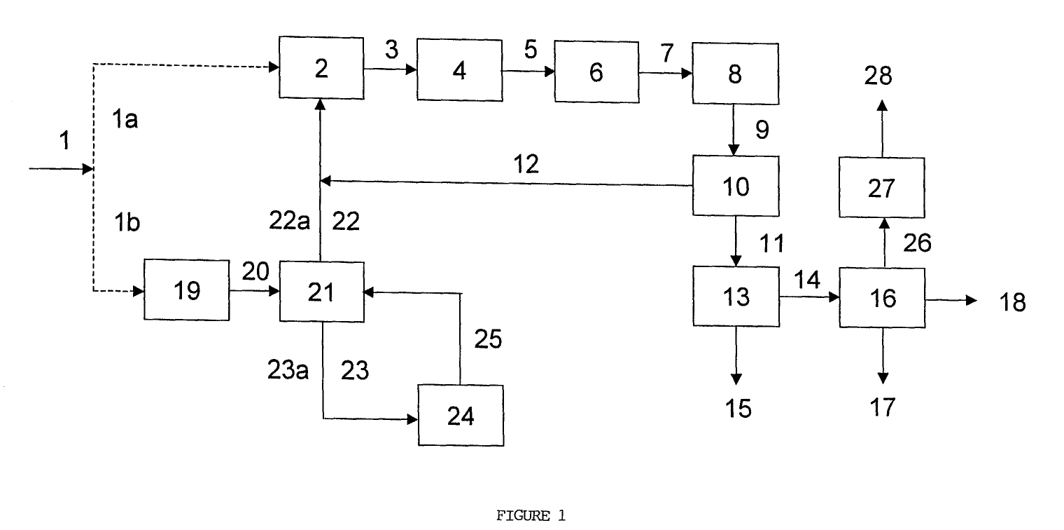

(a) dehydrogenating n-butane and iso-butane present in the field butanes 1 in a dehydrogenation stage 2;

(b) oligomerizing the dehydrogenation mixture 3 in an oligomerization stage 8;

(c) separating di-iso-butene, di-n-butene and residual gas from the oligomerization mixture; and

(d) hydrogenating di-iso-butenes to give di-iso-butanes.

### BACKGROUND OF THE INVENTION

1. Field of the Invention

The present invention relates to a process for preparing butene oligomers, which are valuable starting materials for plasticizer alcohols, from field butanes. Preferred butene oligomers are the isomeric octenes which are dimeric butenes and are, therefore, also termed di-butene. Di-butenes particularly in demand are di-n-butene and di-iso-butene. More preferably, the invention relates to a process in which di-iso-butene is separated from di-butene and further processed.

2. Description of the Background

Di-butene is an isomeric mixture which is formed, in addition to higher butene oligomers, by dimerization and/or codimerization of butenes, i.e. of n-butene and/or isobutene, in the oligomerization of butenes. The term di-n-butene is applied to the dimerization product of n-butene, i.e. of 1-butene and/or of 2-butene. Important components of di-n-butene are 3-methyl-2-heptene, 3,4-dimethyl-2-hexene and, to a lesser extent n-octenes. Di-isobutene is the mixture of dimers which is formed by dimerization of isobutene. Di-isobutene contains molecules which are more highly branched than di-butene, and di-butene in turn is more highly branched than di-n-butene.

Di-butene, di-n-butene and di-iso-butene are starting materials for preparing isomeric nonanols by hydroformylation and hydrogenation of the C₉ aldehydes thus formed. Esters of these nonanols, in particular the phthalic esters, include plasticizers, which are prepared to a significant extent, and are primarily used for polyvinyl chloride. Nonanols, prepared from di-n-butene, are linear to a greater extent than nonanols prepared from di-butene, which are in turn less branched than nonanols prepared from diisobutene. Esters of nonanols prepared from di-n-butene, because of their more linear structure, have advantages in application in comparison to esters prepared from nonanols based on di-butene and di-isobutene and are particularly in demand.

Butenes can be obtained for use as starting material in the dimerization reaction from the C₄ fraction of steam crackers or of FC crackers, for example. This fraction is generally worked-up, by first separating 1,3-butadiene by a selective scrubbing, e.g. by scrubbing with n-methylpyrrolidone. Isobutene is a desirable and particularly valuable C₄ fraction component, because it may be chemically reacted to give sought-after products, e.g. with iso-butane, to give high-octane isooctane or with methanol to give methyl tert-butyl ether (MTBE), which, as an additive in motor gasoline, improves its octane rating. After the reaction of the isobutene, the n-butenes and n-butane and iso-butane remain behind. The proportion of n-butenes in the cracked products of the steam cracker or the FC cracker is relatively low, however, that is, on the order of magnitude of barely 10% by weight, based on the principal target product ethylene. A steam cracker having the respectable capacity of 600,000 metric t/year of ethylene, therefore, only delivers around 60,000 metric t/year of n-butene. Although the amount of n-butene (and that of the isobutenes) could be increased by dehydrogenating the around 15,000 metric t/year of n-butane and iso-butane which arise in addition to the n-butenes, this is not advisable, because dehydrogenation plants require high capital expenditure and are uneconomic for such a small capacity.

Isobutene is, as stated, a cracked product in demand and is, therefore, not generally available for the oligomerization. The amount of n-butenes which a steam cracker or an FC cracker produces directly is not sufficient, however, to produce sufficient di-butene for a nonanol plant whose capacity is so high that it could compete economically with the existing large-scale plants for preparing important plasticizer alcohols, such as 2-ethylhexanol. N-butenes from various steam crackers or FC crackers would, therefore, have to be collected and oligomerized together, in order to cover the di-butene demand of a large nonanol plant. Opposing this, however, is the fact that the transport of liquified gases is expensive, not least because of the complex safety precautions required.

It would, therefore, be desirable if butenes could be provided at only one site without transport over relatively large distances in amounts for the oligomerization reaction as are required for the operation of a large scale plant for preparing nonanols, for example, having a capacity of 200,000 to 800,000 metric t/year. It would further be desirable to have a process for preparing butene oligomers in which the valuable di-iso-butene can be separated from the di-butene. The di-iso-butenes can be hydrogenated to yield di-iso-butanes, which are valuable fuel additives as a substitute for methyl-tert-butyl-ether. Finally, it would be desirable if the process could be controlled in such a manner that, in addition to higher butene oligomers, only di-iso-butene is formed as di-butene.

### SUMMARY OF THE INVENTION

Accordingly, one object of the present invention is to provide a process of effectively providing di-iso-butenes and di-iso-butanes from field butanes which provides the desired products simply on site in quantities desired.

Briefly, this object and other objects of the present invention as hereinafter will become more readily apparent can be attained by a process for preparing oligomers and di-iso-butanes from field butanes which comprises:

(a) dehydrogenating the n-butane and isobutane present in the field butanes 1 in a dehydrogenation stage 2,

(b) oligomerizing the dehydrogenation mixture 3 in an oligomerization stage 8 to give an oligomerization mixture 9,

(c) separating di-iso-butene, di-n-butene and residual gas from the oligomerization mixture, and

(d) hydrogenating di-iso-butenes to give di-iso-butanes.

### BRIEF DESCRIPTION OF THE DRAWING

A more complete appreciation of the invention and many of the attendant advantages thereof will be readily obtained as the same becomes better understood by reference to the following detailed description when considered in connection with the accompanying drawing, wherein the FIGURE shows the scheme of Variants A and B of the invention.

### DETAILED DESCRIPTION OF THE PREFERRED EMBODIMENTS

The process of the invention is described in more detail by the block diagram of the accompanying FIGURE, in which the Variants A and B, described in more detail below, are shown together with their obligatory and optional process stages. Field butane 1 is assigned as steam 1a in Variant A and the alternative stream 1b is a part of Variant B.

Variant A is described below.

In detail, di-butene 14 is separated from the oligomers 11, which remain after separating the residual gases 12 from the oligomerization mixture 9. After separation of the di-butene 14, di-n-butene 17, and/or di-iso-butene 26 can be obtained separately by means of the distillation stage 16.

In Variant B, the process can be controlled in such a manner that, in addition to higher butene oligomers, only di-iso-butene is formed, by separating isobutane 22a by fractional distillation from the, if appropriate, previously hydrogenated, field butane 1, isomerizing the remaining n-butane 23a in an isomerization stage 24 to give a mixture of n-butane and iso-butane, separating the iso-butane from the isomerization mixture 25 by fractional distillation, and conducting the iso-butane into the dehydrogenation stage 2, together with the iso-butane 22a separated directly from the field butane 1, and recycling the remaining n-butane 23a into the isomerization stage 24.

The process of the invention with its Variants A and B is distinguished by high flexibility. Therefore, depending on market requirements, if desired, only di-n-butene, di-butene, di-iso-butene, and other di-butenes conjointly or only di-iso-butene in addition to di-iso-butane cane be produced.

The term field butanes is applied to the C₄ fraction of the “moist” portions of natural gas and of crude oil-associated gases which are separated from the gases in liquid form by cooling to about −30° C. Low-temperature distillation produces the field butanes therefrom, whose composition fluctuates with the field, but which generally contain about 30% iso-butanes and 65% n-butane. Further components are generally about 2% C_{<4} hydrocarbons and about 3% C_{>4} hydrocarbons. Field butanes can be used without separation as feedstuffs in steam crackers or as an additive to motor gasoline. They may be resolved into n-butane and iso-butane by fractional distillation, Iso-butane is used, e.g., to an important extent for preparing propylene oxide by cooxidation of propylene and iso-butane and is also used as an alkylating agent, by which n-butene or isobutene is alkylated to give isooctane which is valued as an additive to motor gasoline because of its high octane rating. In contrast, n-butane has only found less important uses. It serves, e.g., as butane gas for heating purposes or is used in relatively small amounts, e.g. for preparing polymers or copolymers or maleic anhydride by atmospheric oxidation. Formerly, n-butane was also dehydrogenated via the n-butene stage to give 1,3-butadiene, but this process has become uneconomic in the interim.

Because iso-butane is the more sought-after component of the field butane, n-butane is isomerized on a large scale to iso-butane (cf., e.g. R. A. Pogliano et. al., Dehydrogenation-based Ether Production, 1996 Petrochemical Review. DeWitt & Company, Houston, Tex., Butame® process, page 6; and S. T. Bakas, F. Nierlich et al., Production of Ethers from Field Butanes and Refinery Streams, AIChE Summer Meeting, 1990, San Diego, Calif., page 11).

Variant A

The field butanes 1a are first dehydrogenated in the dehydrogenation stage 2. The dehydrogenation is a codehydrogenation. It is remarkable that the dehydrogenation of the field butane, which is a mixture of components having different dehydrogenation behaviors, succeeds so readily. The process conditions substantially correspond to those which are known for n-butane and iso-butane or other lower hydrocarbons. Thus, S. T. Bakas, F. Nierlich et al., loc. cit., pages 12 ff., describe the Oleflex® process which is generally suitable for the selective preparation of light olefins and by which iso-butane can be dehydrogenated to isobutene with a selectivity of 91 to 93%. Further relevant publications are those of G. C. Sturtevant et al., Oleflex—Selective Production of Light Olefins, 1988 UOP Technology Conference and EP 0 149 698. The dehydrogenation is expediently conducted in the gas phase on fixed-bed or fluidized catalysts, e.g. on chromium (III) oxide or, advantageously, on platinum catalysts having aluminum oxide or zeolites as support. The dehydrogenation generally takes place at temperatures of 400 to 800° C., advantageously from 550 to 650° C.

Atmospheric pressure or a slightly elevated pressure of up to 3 bar is generally employed. The residence time in the catalyst bed generally ranges from 1 to 60 minutes, depending on catalyst, temperature and desired degree of conversion. The throughput accordingly generally ranges from 0.6 to 36 kg of field butane per m³ of catalyst and hour.

It is expedient to conduct the dehydrogenation only until about 50% of the n-butane and iso-butane remain unchanged in the dehydrogenation mixture 3. Although higher degrees of conversion can be attained at higher temperature, cracking reactions which decrease the yield proceed to an increasing extent, because of coke deposits, which reduce the service life of the dehydrogenation catalyst. The optimum combinations of the reaction conditions which lead to the desired degrees of conversion, such as type of catalyst, temperature and residence time, may be determined without difficulty by preliminary experiments.

The dehydrogenation mixture 3 generally contains 90 to 95% C₄ hydrocarbons and, in addition, hydrogen and lower- and higher-boiling portions which in part originate from the field butane 1, and in part are formed in the dehydrogenation stage 2. Purification is expediently performed upstream of the oligomerization. In a first purification stage (not shown in the FIGURE), the C₄ fraction and the higher-boiling portions are removed by condensation. The condensate is distilled under pressure, co-condensed dissolved C_{<4} hydrocarbons passing overhead. From the bottom product, in a further distillation the C₄ hydrocarbons are obtained as main product and the comparatively small amount of C_{>4} hydrocarbons is obtained as residue.

The C₄ hydrocarbons, depending on the degree of conversion, generally contain small amounts, such as 0.01 to 5% by volume, of 1,3-butadiene. It is advisable to remove this component since, even in markedly lower amounts, it can damage the oligomerization catalyst. A suitable process is selective hydrogenation 4 which, in addition, increases the proportion of the desired n-butene. A suitable process has been described, e.g., by F. Nierlich et. al. in Erdöl & Kohle, Erdgas, Petrochemie, 1986, pages 73 ff. It operates in the liquid phase with completely dissolved hydrogen in stoichiometric amounts. Selective hydrogenation catalysts which are suitable include nickel and, in particular, palladium, on a support, e.g. 0.3% by weight of palladium on activated carbon or, preferably, on aluminum oxide. A small amount of carbon monoxide in the ppm range promotes the selectivity of the hydrogenation of 1,3-butadiene to give the monoolefin and counteracts the formation of polymers, the so-called “green oil”, which inactivate the catalyst. The process generally operates at room temperature or elevated temperatures up to about 60° C. and at elevated pressures which are expediently in the range of up to 20 bar. The content of 1,3-butadiene in the C₄ fraction of the dehydrogenation mixture is decreased in this manner to values of <1 ppm.

It is also expedient to pass the dehydrogenation mixture 5 C₄ fraction, which is then substantially freed from 1,3-butadiene, via the purification stage 6, a molecular sieve, upstream of the oligomerization stage, as a result of which more of the substances which are harmful to the oligomerization catalyst are removed and its service life is further increased. These harmful substances include oxygen compounds and sulfur compounds. This process has been described by F. Nierlich et al. in EP-B1 0 395 857. A molecular sieve having a pore diameter of 4 to 15 angstroms, advantageously 7 to 13 angstroms, is expediently used. In some cases it is expedient for economic reasons to pass the dehydrogenation mixture successively over molecular sieves having different pore sizes. The process can be conducted in the gas phase, in the liquid phase or in the gas-liquid phase. The pressure is accordingly generally 1 to 200 bar. Room temperature or elevated temperatures up to 200° C. are expediently employed.

The chemical nature of the molecular sieves is less important than their physical properties, i.e. in particular their pore size. The most diverse molecular sieves can, therefore, be used, both crystalline natural aluminum silicates, e.g. sheet lattice silicates, and synthetic molecular sieves, e.g. those having a zeolite structure. Zeolites of the A, X and Y type are available, inter alia, from Bayer AG, Dow Chemical Co., Union Carbide Corporation, Laporte Industries Ltd., and Mobil Oil Co. Suitable synthetic molecular sieves for the process are also those which, in addition to aluminum and silicon, contain other atoms introduced by cation exchange, such as gallium, indium or lanthanum, as well as nickel, cobalt, copper, zinc or silver. In addition, synthetic zeolites are suitable in which, in addition to aluminum and silicon, other atoms, such as boron or phosphorus, have been incorporated into the lattice by mixed precipitation.

As already stated, the selective hydrogenation stage 4 and the purification stage 6 using a molecular sieve are optional, advantageous measures for the process according to the invention. Their order is in principle optional, but the order specified in the FIGURE is preferred.

The dehydrogenation mixture 7, if appropriate pretreated in the described manner, is passed into the oligomerization stage 8 which is an essential part of the process of the invention. The oligomerization is a co-oligomerization of n-butenes and isobutene which is conducted in a manner known per se, such as has been described, e.g., by F. Nierlich in Oligomerization for Better Gasoline, Hydrocarbon Processing, 1992, pages 45 ff, or by F. Nierlich et al. in the previously mentioned EP-B1 0 395 857. The procedure is generally conducted in the liquid phase and, as homogeneous catalyst, a system is employed, e.g., which comprises nickel (II) octoate, ethylaluminum chloride and a free fatty acid (DE-C 28 55 423), or preferably one of the numerous known fixed-bed catalysts or catalysts suspended in the oligomerization mixture which are based on nickel and silicon. The catalysts frequently additionally contain aluminum. Thus, DD-PS 160 037 describes the preparation of a nickel- and aluminum-containing precipitated catalyst on silicon dioxide as support material. Other useful catalysts are prepared by exchanging positively charged particles, such as protons or sodium ions, which are situated on the surface of the support materials, for nickel ions. This is successful with the most diverse support materials, such as amorphous aluminum silicate (R. Espinoza et al., Appl. Kat., 31 (1987) pages 259-266; crystalline aluminum silicate (DE-C 20 29 624; zeolites of the ZSM type (NL Patent 8 500 459; an X zeolite (DE-C 23 47 235); X and Y zeolites (A. Barth et al., Z. Anorg. Allg. Chem. 521, (1985) pages 207-214); and a mordenite (EP-A 0 281 208).

The co-oligomerization is expediently conducted, depending on the catalyst, at 20 to 200° C. and under pressures of 1 to 100 bar. The reaction time (or contact time) generally ranges from 5 to 60 minutes. The process parameters, in particular the catalyst type, the temperature and the contact time, are matched to one another in such a manner that the desired degree of oligomerization is attained. In the case of nonanols as desired target product, this is predominantly a dimerization. For this purpose, clearly the reaction must not proceed to full conversion, but conversion rates of 30 to 70% per pass are expediently desired. The optimum combinations of the process parameters may be determined without difficulties by preliminary experiments.

The residual gas 12 is separated from the oligomerization mixture 9 in a separation stage 10 and recycled to the dehydrogenation stage 2. If a catalyst of the liquid catalyst type mentioned is used in the oligomerization stage 8, the residual gas 12 should be purified in advance to protect the dehydrogenation catalyst. The oligomerization mixture is initially treated with water, in order to extract the catalyst components. The residual gas 12 which separates is then dried with a suitable molecular sieve, other minor components also being separated and removed. Then polyunsaturated compounds, such as butynes, are removed by selective hydrogenation, e.g. on palladium catalysts, and the residual gas 12 thus purified is recycled into the dehydrogenation stage 2. These measures for purifying the residual gas 12 are unnecessary if a solid oligomerization catalyst is used.

The oligomers 11 remaining after separation of the residual gas 12 are suitable, because of their branched components, as an additive to motor gasoline to improve the octane rating.

The oligomers 11 are separated in the distillation stage 13 into di-butenes 14 and trimers 15, i.e. isomeric dodecenes, and yet higher oligomers, the main fraction comprising the desired di-butenes 14. The dodecenes 15 can be hydroformylated. The hydroformylation products can be hydrogenated and the tridecanols thus obtained can be ethoxylated, as a result of which valuable detergent bases are obtained.

The di-butenes 14 are separated in the fine distillation stage 16 into di-n-butene 17, di-iso-butenes 26, and the residual di-butenes 18. Since the more highly branched di-butene molecules are lower boiling, the residual di-butenes can likewise be used to prepare nonanols or can be added prior or after hydrogenation to motor gasoline. This procedure is a more expedient alternative to the variant in which n-butene and isobutene are separated from the co-dehydrogenation mixture 7 by distillation and these isomers are oligomerized separately. This variant would require two separate oligomerization stages, which would be considerably more capital-intensive and also more complex in operation than only one, all-be-it larger, co-oligomerization stage 8 in combination with a fine distillation stage 16.

Variant B

This variant is selected when it is desired to prepare only di-iso-butene as dibutene. If the field butane 1 b contains olefinically unsaturated components, it is advantageously first passed into a hydrogenation stage 19, because these components can interfere with the later isomerization of the iso-butane. The hydrogenation proceeds in a manner known per se, such as described by K. H. Walter et al., in the Hüls Process for Selective Hydrogenation of Butadiene in Crude C4's, Development and Technical Application, DGKM meeting Kassel, November 1993. The procedure is, therefore, expediently conducted in the liquid phase and, depending on the catalyst, at room temperature or elevated temperature up to 90° C. and at a pressure of 4 to 20 bar, the partial pressure of the hydrogen being 1 to 15 bar. The catalysts customary for the hydrogenation of olefins are used, e.g. 0.3% palladium on aluminum oxide.

The hydrogenated field butanes 20 are passed into the separation stage 21. This generally comprises a highly effective column in which n-butane 22 and isobutane 23 are separated by fractional distillation. The column 21 is operated in a customary manner, expediently at a pressure ranging from 4 to 7 bar. The isomerization of n-butane and iso-butane is a known reaction (see, e.g., H. W. Grote, Oil and Gas Journal, 56 (13 pages 73 ff., (1958)). The procedure is generally conducted in the gas phase, expediently at a temperature of 150 to 230° C. at a pressure of 14 to 30 bar and over a platinum catalyst on aluminum oxide as support, whose selectivity can be further improved by doping with a chlorine compound, such as carbon tetrachloride. Advantageously, a small amount of hydrogen is added, in order to counteract a dehydrogenation. The selectivity of the isomerization to iso-butane is high; cracking to form smaller fragments only takes place to a minor extent (approximately 2%).

The isomerization mixture 25 must be separated into the isomers. This is expediently performed in column 21 which is present in any case, from which passes into the dehydrogenation stage 2 which, in contrast to Variant A, is not a co-dehydrogenation stage. The isobutane 22a is passed from the column 21 into the dehydrogenation stage 2, in which no co-dehydrogenation occurs. The n-butane 23a is passed from the column 21 into the isomerization stage 24 and there isomerized to at most up to equilibrium. The iso-butane is separated from n-butane, expediently in the column 21, and passed into the dehydrogenation stage 2, whereas the n-butane returns to the isomerization stage 24. In this manner, the n-butane is completely converted into iso-butane. The dehydrogenation mixture 3 is expediently purified as described in Variant A. The oligomerization in the oligomerization stage 8 is a homo-oligomerization, because only iso-butene participates therein, and di-isobutene occurs in distillation stage 13. The fine distillation 16 is likewise omitted.

In both variants A and B, the resulting di-iso-butenes 26 can be hydrogenated in stage 27 to give di-iso-butane 28. Both compounds, 26 and 28, can be used as fuel additives to increase octane rating of fuel. The hydrogenation stage 27 can be performed as known in the art e.g. via a nickel-containing catalyst or in the same manner as the hydrogenation stage 19.

The disclosure of European priority Application No. 001 16504.2 filed Jul. 31, 2000 is hereby incorporated by reference into the present application.

Obviously, numerous modifications and variations of the present invention are possible in light of the above teachings. It is, therefore, to be understood that within the scope of the appended claims, the invention may be practiced otherwise than as specifically described herein.

What is claimed as new and is intended to be secured by Letters Patents is:

1. A process for preparing butene oligomers and di-iso-butane from field butanes, which comprises:
(a) dehydrogenating n-butane and iso-butane present in the field butanes 1 in a dehydrogenation stage 2;
(b) oligomerizing the dehydrogenation mixture 3 in an oligomerization stage 8;
(c) separating di-iso-butene, di-n-butene and residual gas from the oligomerization mixture; and
(d) hydrogenating di-iso-butenes to give di-iso-butanes.

2. The process as claimed in claim 1, which further comprises, between dehydrogenating step (a) and oligomerizing step (b), in any order, selectively hydrogenating 4 the product of step (a) and/or purifying 6 the product of step (a) with a molecular sieve.

3. The process as claimed in claim 1, comprising separating residual gas 12 from the oligomerization mixture 9 and, optionally, after purification, recycling the gas into the dehydrogenation stage 2.

4. The process as claimed in claim 2, comprising separating residual gas 12 from the oligomerization mixture 9 and, optionally, after purification, recycling the gas into the dehydrogenation stage 2.

5. The process as claimed in claim 3, comprising separating di-butene 14 from the oligomers 11 remaining after separating the residual gas 12 from the oligomerization mixture 9.

6. The process as claimed in claim 4, comprising separating the di-butenes 14 in a fine distillation stage 16 into di-n-butenes 17, di-iso-butene 26 and residual di-butenes 18.

7. The process as claimed in claim 1, comprising, separating iso-butane from the optionally prehydrogenated field butane 1 by fractional distillation and passing the separated iso-butane into the dehydrogenation stage 2, isomerizing remaining n-butane in an isomerization stage 24 to give a mixture of n-butane and iso-butane, separating the isobutane from the isomerization mixture 25 by fractional distillation and conducting iso-butane into the dehydrogenation stage 2 together with the iso-butane separated directly from the field butane, and recycling remaining n-butane into the isomerization stage 24, thereby producing only di-iso-butene 26.

8. The process as claimed in claim 1, wherein the dehydrogenation of field butanes is conducted over a fluidized or fixed bed catalyst at 400-800° C. and under a pressure of atmospheric to 3 bar.

9. The process as claimed in claim 8, wherein the dehydrogenation is conducted until only about 50% of the n-butane and iso-butane remains unchanged in dehydrogenation mixture 3.

10. The process as claimed in claim 1, wherein, prior to oligomerization in step (b), the dehydrogenated material of step (a) is purified over a molecular sieve to remove butadiene therefrom.

11. The process as claimed in claim 10, wherein the molecular sieve is a crystalline natural silicate or a synthetic molecular sieve.

12. The process as claimed in claim 10, wherein the oligomerization of the dehydrogenation mixture 3 comprising n-butenes and isobutene is conducted in the liquid phase in the presence of a homogeneous catalyst, a nickel- and aluminum-containing precipitated catalyst on silicon dioxide, amorphous aluminum silicate, crystalline aluminum silicate, X and Y zeolites or a mordenite.

13. The process as claimed in claim 10, wherein the oligomerization of the dehydrogenation mixture 3 comprising n-butenes and isobutene is conducted in the presence of a catalyst at 20 to 200° C. under a pressure of 1 to 100 bar.

14. The process as claimed in claim 1, wherein the residual gas is recycled to the dehydrogenation stage 2.

15. The process as claimed in claim 1, wherein the di-butenes of step (c) are separated into di-n-butene, di-iso-butenes and residual di-butenes.

16. A method of preparing nonanols, comprising:
hydroformylating a mixture of di-n-butenes 16 and residual di-butenes prepared by the process of claim 1; and then
hydrogenating the hydroformylation product to prepare the nonanols.

17. A method of preparing nonanoic acids, comprising:
hydroformylating di-n-butenes 16 prepared by the process of claim 1; and then
oxidizing the hydroformylation product to prepare said nonanoic acids.

18. A method of formulating hydrocarbon fuel, comprising:
adding the di-iso-butane product prepared by the process of claim 1 to a hydrocarbon fuel base.
